SEQUENCE LISTING (23 sequences)

SEQ ID NO: 1 - protein, 9 aa, Human immunodeficiency virus type 1
  MISC_FEATURE (4)..(4): Xaa can be Arg, Lys, Ser or Asn
VDPXLXPWX

SEQ ID NO: 2 - protein, 11 aa, Human immunodeficiency virus type 1
  MISC_FEATURE (2)..(2): Xaa can be Gly or Ala
KXLGISYGRKK

SEQ ID NO: 3 - protein, 7 aa, Human immunodeficiency virus type 1
  MISC_FEATURE (3)..(3): Xaa can be Ala, Pro, Ser or Gln
RRXXXXS

SEQ ID NO: 4 - protein, 12 aa, Human immunodeficiency virus type 1
  MISC_FEATURE (3)..(3): Xaa can be Asn or Thr
SQXHQXSLSKQP

SEQ ID NO: 5 - protein, 10 aa, Human immunodeficiency virus type 1
  MISC_FEATURE (2)..(2): Xaa can be Gly or Ala
KXLGISYGRK

SEQ ID NO: 6 - protein, 14 aa, Human immunodeficiency virus type 1
  MISC_FEATURE (1)..(1): Xaa can be absent or Val or Glu
XXDPXLXPXXXXXX

SEQ ID NO: 7 - protein, 8 aa, Human immunodeficiency virus type 1
  MISC_FEATURE (5)..(5): Xaa can be Arg, Lys, Ser or Asn
EVDPXLXP

SEQ ID NO: 8 - protein, 8 aa, Human immunodeficiency virus type 1
  MISC_FEATURE (4)..(4): Xaa can be Arg, Lys, Ser or Asn
VDPXLXWX

SEQ ID NO: 9 - protein, 12 aa, Human immunodeficiency virus type 1
  MISC_FEATURE (4)..(4): Xaa can be Arg, Lys, Ser or Asn
VDPXLXWXHPGS

SEQ ID NO: 10 - protein, 7 aa, Human immunodeficiency virus type 1
VDPRLEP

SEQ ID NO: 11 - protein, 7 aa, Human immunodeficiency virus type 1
VDPKLEP

SEQ ID NO: 12 - protein, 7 aa, Human immunodeficiency virus type 1
VDPSLEP

SEQ ID NO: 13 - protein, 7 aa, Human immunodeficiency virus type 1
VDPNLEP

SEQ ID NO: 14 - protein, 7 aa, Human immunodeficiency virus type 1
VDPRLDP

SEQ ID NO: 15 - protein, 7 aa, Human immunodeficiency virus type 1
VDPKLDP

SEQ ID NO: 16 - protein, 7 aa, Human immunodeficiency virus type 1
VDPSLDP

SEQ ID NO: 17 - protein, 7 aa, Human immunodeficiency virus type 1
VDPNLDP

SEQ ID NO: 18 - protein, 10 aa, Human immunodeficiency virus type 1
KGLGISYGRK

SEQ ID NO: 19 - protein, 10 aa, Human immunodeficiency virus type 1
KALGISYGRK

SEQ ID NO: 20 - protein, 17 aa, Human immunodeficiency virus type 1
  MISC_FEATURE (2)..(2): Xaa can be Gly or Ala
KXLGISYGRKKRRQRRR

SEQ ID NO: 21 - protein, 4 aa, Human immunodeficiency virus type 1
HPGS

SEQ ID NO: 22 - protein, 7 aa, Human immunodeficiency virus type 1
  MISC_FEATURE (4)..(4): Xaa can be Arg, Lys, Ser or Asn
VDPXLXP

SEQ ID NO: 23 - protein, 8 aa, Human immunodeficiency virus type 1
  MISC_FEATURE (3)..(3): Xaa can be Arg, Lys, Ser or Asn
DPXLXPWX

(19) United States
(12) Patent Application Publication
(10) Pub. No.: US 2008/0280305 A1
(43) Pub. Date: Nov. 13, 2008

(54) Assays for Detecting Hiv-1 Tat Protein in Hiv-1 Infection

(75) Inventor: Gideon Goldstein, Short Hills, NJ (US)

Correspondence Address: HOWSON AND HOWSON, SUITE 210, 501 OFFICE CENTER DRIVE, FT WASHINGTON, PA, 19034, US

(73) Assignee: Thymon, LLC, Short Hills, NJ (US)

(21) Appl. No.: 11/795,671

(22) PCT Filed: Feb. 13, 2006
(86) PCT No.: PCT/US2006/004781

§ 371 Date: Jul. 19, 2007

Related U.S. Application Data
(60) Provisional application No. 60/652,928, filed on Feb. 15, 2005.

(51) Int. Cl. G01N 33/53

(52) U.S. Cl. 435/7.1

(57) ABSTRACT

A method for detecting or measuring HIV-1 Transactivating (Tat) protein in a biological sample comprising contacting the biological sample with an amount of a basic protein effective to reduce interference with binding between anti-HIV-1 Tat Epitope 2 ligand and Tat that is caused by acidic components within the sample or reagents. A more accurate detection and measurement of the amount of HIV-1 Tat in the sample is obtained by binding between the anti-Epitope 2 antibody and the Tat in the sample when the interference is neutralized. A diagnostic kit for use in practicing the method contains these components.

### BACKGROUND OF THE INVENTION

The present invention relates generally to compositions and methods useful for diagnosis of human immunodeficiency virus-1 (HIV-1) infection in patients, symptomatic or asymptomatic, by detecting the presence and measuring the amount of the HIV-1 Tat protein and reducing interference with assay results caused by substances in the biological fluids assayed.

Methods for early detection of HIV-1 infection, particularly for the screening of blood products and the blood supply, as well as patient diagnosis, are highly desirable. However, to date, the earliest detectable marker of HIV-1 infection is the presence of HIV-1 viral RNA. Currently employed diagnostic assays detect the viral RNA by branched DNA (bDNA; e.g., HIV-1 b-DNA assay, Chiron) or polymerase chain reaction (PCR; e.g., HIV-1 Monitor™ PCR Assay, Roche Diagnostics) techniques. Another assay for diagnosis of HIV-1 infection includes an immunoassay to detect the HIV-1 p24 protein, which is commonly detectable within 1-3 days after viral RNA is detectable. Still another assay detects antibodies to HIV-1 virion proteins (seroconversion), which is usually not detectable before 7 days after detection of viral RNA. An assay for detecting circulating HIV-1 transactivating (Tat) protein in plasma was previously described, but found to be relatively insensitive (Westendorp et al, 1995 Nature, 375:497-500).

The transactivating (tat) gene of HIV-1 expresses a protein (Tat) essential for high levels of transcription of the virus. The tat gene and its protein have been sequenced and examined for involvement in proposed treatments of HIV (see, e.g., the documents cited in U.S. Pat. No. 6,525,179). Tat protein is released extracellularly, making it available to be taken up by other infected cells to enhance transcription of HIV-1 in the cells and to be taken up by noninfected cells, altering host cell gene activations and rendering the cells susceptible to infection by the virus. Uptake of Tat by cells is very strong, and has been reported as mediated by a short basic sequence of the protein (S. Fawell et al., 1994 Proc. Natl. Acad. Sci., USA, 91:664-668).

Both monoclonal and polyclonal antibodies to Tat protein have been readily produced in animals and shown to block uptake of Tat protein in vitro. Such monoclonal or polyclonal antibodies to Tat protein added to tissue culture medium have attenuated HIV-1 infection in vitro (see, e.g., documents cited in U.S. Pat. No. 6,524,582, which is incorporated by reference herein).

Prior scientific publications and patent publications by the present inventor (e.g., G. Goldstein, 1996 Nature Med., 2:960; G. Goldstein, 2000 Vaccine, 18:2789; International Patent Publication No. WO95/31999, published Nov. 30, 1995; International Patent Publication No. WO99/02185, published Jan. 21, 1999; International Patent Publication No. WO 01/82944, published Nov. 8, 2001; U.S. Pat. Nos. 5,891,994; 6,193,981; 6,399,067; 6,524,582; 6,525,179; US Published Patent Application Nos. US 2003/0166,832 and US 2003/0,180,326, all incorporated by reference herein) refer to antibodies to certain epitopes on HIV-1 Tat protein.

For example, these publications refer to antibodies which specifically bind to an epitope located within the “HIV-1 Tat Epitope 1” sequence spanning Tat amino acid residues 4-12, as follows: Val-Asp-Pro-X₇-Leu-Y₉-Pro-Trp-Z₁₂- SEQ ID NO: 1, wherein X₇ is Arg, Lys, Ser or Asn, Y₉ is Glu or Asp, and Z₁₂ is Lys or Asn, and compositions combining this antibody with other antibodies to HIV-1 Tat. These publications also refer to another antibody composition containing isolated antibodies which bind specifically to the “HIV-1 Tat Epitope 2” sequence located within Tat amino acid residues 41-51 of the formula -Lys-X₄₂-Leu-Gly-Ile-Ser-Tyr-Gly-Arg-Lys-Lys- SEQ ID NO: 2, where X₄₂ is Gly or Ala. Additionally, these publications refer to isolated antibodies which bind specifically to the “HIV-1 Tat Epitope 3” sequence located within Tat amino acid residues 56-62 of the formula -Arg-Arg-X₅₈-Z₅₉-A₆₀-Y₆₁-Ser- SEQ ID NO: 3, wherein X₅₈ is Ala, Pro, Ser, or Gln, Z₅₉ is Pro or His, A₆₀ is Gln or Pro, and Y₆₁ is Asp, Asn, Gly or Ser, or to the “HIV-1 Tat Epitope 4” sequence located within Tat amino acid residues 62-73 of the formula -Ser-Gln-X₆₄-His-Gln-Y₆₇-Ser-Leu-Ser-Lys-Gln-Pro- SEQ ID NO: 4, wherein X₆₄ is Asn or Thr, and Y₆₇ is Ala or Val.

Compositions formed of combinations of these antibodies, particularly combinations of an antibody that specifically binds one Epitope 1 variant with one or more antibodies that each specifically bind a different Epitope 1 variant, and further combinations of such Epitope 1 antibodies with an Epitope 2 antibody, among other combinations, are able to bind a large number of Tat variant sequences characteristic of the multiple strains and subtypes of HIV-1, both B and non-B clades. These antibody compositions or mixtures of such anti-Tat antibodies are advantageous as diagnostic agents able to detect many strains and subtypes of the virus, thus obviating the need for different and strain-specific diagnostic agents.

Despite the current diagnostic assays for detection of HIV-1, there remains a need in the art for the development of additional assays for simple and accurate screening and diagnosis of HIV-1 infection.

### SUMMARY OF THE INVENTION

In one aspect, the present invention provides a method or diagnostic assay useful for diagnosis of HIV-1 infection through detection of the HIV-1 Transactivating (Tat) protein produced by the virus in a biological sample. The method involves introducing an amount of a basic protein into assay reagents and/or the sample being assayed. The basic protein is present in an amount or concentration relative to the sample undergoing assay or reagent to be used in the assay, which amount is effective to prevent acidic proteins or other acidic substances within the sample or reagent from interfering with binding between a ligand to Tat Epitope 2 and the Tat in the sample.

In one particular embodiment, the invention provides a method or diagnostic assay comprising the steps of contacting the biological sample with (i) an antibody or antibody fragment that specifically binds to HIV-1 Tat protein within the Epitope 2 amino acid sequence Lys-X-Leu-Gly-Ile-Ser-Tyr-Gly-Arg-Lys- SEQ ID NO: 5, wherein X is Gly or Ala, and (ii) an amount of a basic protein effective to reduce interference with binding between that anti-Epitope 2 antibody and the Tat by acidic proteins within the sample. The method further determines the presence or amount of HIV-1 Tat in the sample by the presence or amount of binding between the anti-Epitope 2 antibody and the Tat in the sample. In one embodiment, such an assay is a sandwich assay employing a capture and a detector antibody, both binding to different epitopes on HIV-1 Tat. In a specific embodiment, the capture antibody is the anti-Epitope 2 antibody and the detector antibody is an antibody or mixture of different antibodies that bind HIV-1 Tat Epitope 1, as defined herein.

In another aspect, the invention provides a kit for use in performing a diagnostic assay on a biological sample, which contains, among other components, a ligand to Tat Epitope 2 (e.g., an anti-Epitope 2 antibody or antibody fragment that binds to an HIV-1 Tat epitope within the amino acid sequence Lys-X-Leu-Gly-Ile-Ser-Tyr-Gly-Arg-Lys- SEQ ID NO: 5, wherein X is Gly or Ala), an optional second antibody or antibody fragment that binds to an HIV-1 Tat epitope different from that of the anti-Epitope 2 antibody, and a basic protein component to inhibit interference in the binding of the anti-Epitope 2 ligand with any Tat in the sample. In one embodiment of the kit, the second antibody is an antibody that specifically binds a variant of HIV-1 Tat Epitope 1. In another embodiment, the kit contains a mixture of antibodies, each antibody specifically binding a different variant of Epitope 1. In still another embodiment, the kit provides any antibody or antibody construct that specifically binds multiple variants of Epitope 1.

Other aspects and advantages of the present invention are described further in the following detailed description of the preferred embodiments thereof.

### BRIEF DESCRIPTION OF THE DRAWINGS

FIG. 1 is a graph of a sandwich assay described in Example 1 below, according to the present invention. A plate was coated with 2 μg/ml anti-Epitope 1 capture antibody and washed with buffer. Samples of 50% plasma with increasing Tat protein concentration were employed as control samples () or test plasma samples indicated by the symbols ∘, □, and ⋄, which were incubated with the basic protein, protamine sulfate at concentrations of 0.05 mg/ml, 0.1 mg/ml and 0.2 mg/ml, respectively, for 1 hour. Each plasma sample was added to a plate. After sufficient time for the Tat in the samples to react with the bound capture antibody, the plates were washed with buffer. Thereafter, detector antibody was then introduced to the plate. Rabbit anti-HIV-1 Tat Epitope 2 antiserum was used as the detector antibody, and was itself pre-incubated with protamine sulfate at the three concentrations indicated. Goat-anti-rabbit IgG, labelled with horseradish peroxidase (HRP), was also added to the plate to provide the signal for the immobilized sandwich. The data shows that the more Tat in the buffer, the more signal is generated due to increased binding of the Tat to the capture antibody, as revealed by the detector antibody-IgG complex in all cases. However, when the basic protein incubation step is employed to neutralize acidic proteins present in the plasma that appear to interfere with binding of the detector antibody to Epitope 2, more sensitive and accurate results are obtained.

FIG. 2 is a graph of dose response curves for a Tat sandwich assay described in Example 2 below and was performed using human plasma, each sample having increasing amounts of HIV-1 Tat. Each sample is incubated with either protamine sulfate (PS) at 0.2 mg/ml (), polyarginine (PA) at 0.2 mg/ml (♦), or polylysine (PL) at 0.2 mg/ml (▪), for 1 hour. The control contained no basic protein (◯). Samples were added to plates which had been coated with capture antibody, which is antibody to Epitope 1 or a mixture of anti-Epitope 1 antibodies (2 μg/ml), and washed. After sufficient time for the Tat in the samples to react with the bound capture antibody, the plates were washed with buffer. The detector antibody is rabbit anti-Epitope 2 antibody, pre-incubated with the same concentrations of the basic protein. The detector antibody is applied to the plates with a goat-anti-rabbit IgG, labelled with HRP, to provide the signal for the immobilized sandwich. The more Tat in the sample, the more signal is generated due to increased binding of the sandwiched IgG complex to the plate in all cases. More sensitive detection is obtained using the basic protein incubation step to neutralize acidic proteins present in the plasma that appear to interfere with binding of the antibody to Epitope 2. All three basic proteins are shown to be useful in this assay, with protamine sulfate being the most active at the indicated concentration.

FIG. 3 is a graph illustrating preliminary data obtained by using the sandwich assay of Example 3 below to measure HIV-1 Tat levels in serial samples in patient sera during early infection. This figure compares the results to first detection of viral RNA (indicated by timepoint 0) by conventional assays currently employed in HIV diagnosis. A total of 20 uninfected controls and 23 samples of serum from asymptomatic HIV-1 infected subjects were also tested. Sixty (60) μg/ml protamine sulfate was added to all control and patient samples for an hour. The capture and detector antibodies and protocol were otherwise as described for FIG. 2. All uninfected controls and 20 of the serum samples from asymptomatic HIV-1 infected subjects recorded Tat concentration at less than 1 ng/ml, i.e., below the dotted line labelled LOD (limit of detection). Three individual subjects were identified by the symbols , ▪ and ♦. As observed by this table, two of these subjects ( and ♦) had serum samples, negative for viral RNA, taken before timepoint 0. These two subjects showed detectable levels of Tat in serum at least 20 days before RNA detection. All three individual subjects showed detectable Tat after timepoint 0.

FIG. 4 is a graph showing HIV RNA levels in the three indicated subjects of FIG. 3 vs. days after first detectable RNA (day 0) as measured by PCR assay (Roche). Subjects ( and ♦) showed increasing viral load typical of acute infection, while subject ▪ appeared to be detected later in the course of infection, showing initial decline of viral load, with no earlier samples available to show true time of appearance of viral RNA.

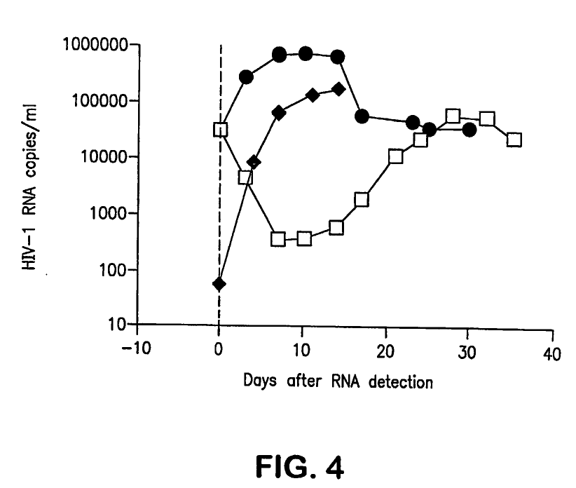

### DETAILED DESCRIPTION OF THE INVENTION

This invention addresses the need in the art for sensitive methods and compositions for use in diagnosing infection with HIV-1 with increased accuracy and sensitivity in biological samples by neutralizing interference in the binding of ligands, such as anti-Tat antibodies, to Tat Epitope 2 caused by acidic proteins in the biological samples. Optionally, the methods and compositions of this invention can result in increasing sensitivity of detection of a full range of HIV-1 strains and subtypes, due to the reactivities of the anti-Epitope 1 antibodies employed in the assay with anti-Epitope 2 antibodies.

### I. Definitions

As used herein, the term “HIV-1 Tat Epitope 1” refers to the sequences represented by the formula

R₁-Asp-Pro-X₇-Leu-Y₉-Pro-R₂ SEQ ID NO: 6,

wherein X₇ is Arg, Lys, Ser or Asn; Y₉ is Glu or Asp; R₁ is absent or Val or Glu-Val; and R₂ is absent or Trp-Z₁₂-R₃, wherein Z₁₂ is absent, Lys or Asn; and R₃ is absent or is all or part of the sequence -His-Pro-Gly-Ser- SEQ ID NO: 21. According to one preferred embodiment, an Epitope 1 sequence contains the variable amino acids at the X₇ and Y₉ positions and an R₁ which is Val, i.e., Val-Asp-Pro-X₇-Leu-Y₉-Pro SEQ ID NO: 22. According to another embodiment an Epitope 1 sequence contains the three variable positions, X₇, Y₉ and Z₁₂ positions and absent R₁, e.g., Asp-Pro-X₇-Leu-Y₉-Pro-Trp-Z₁₂ SEQ ID NO: 23. Given the above formula, the entire scope of variant Epitope 1 sequences may be sequences of between 7 and about 14 amino acids in length, either containing fragments of the above-identified SEQ ID NO: 6 or larger sequences encompassing the fragments or entirety of SEQ ID NO: 6. Thus there exist greater than the eight Epitope 1 variant sequences specified by the Examples below.

Other variant HIV-1 Tat Epitope 1 sequences include the sequences represented by the formula Glu-Val-Asp-Pro-X₇-Leu-Y₉-Pro SEQ ID NO: 7, Val-Asp-Pro-X₇-Leu-Y₉-Trp-Z₁₂- SEQ ID NO: 8, and Val-Asp-Pro-X₇-Leu-Y₉-Trp-Z₁₂-His-Pro-Gly-Ser- SEQ ID NO: 9, as well as other sequences falling within the above formula. In one desirable embodiment, exemplified below, the selected variant HIV-1 Epitope 1 sequences are represented by the following eight sequences:

| | | |
|---|---|---|
| (a) | -Val-Asp-Pro-Arg-Leu-Glu-Pro- | SEQ ID NO: 10 |
| (b) | -Val-Asp-Pro-Lys-Leu-Glu-Pro- | SEQ ID NO: 11 |
| (c) | -Val-Asp-Pro-Ser-Leu-Glu-Pro- | SEQ ID NO: 12 |
| (d) | -Val-Asp-Pro-Asn-Leu-Glu-Pro- | SEQ ID NO: 13 |
| (e) | -Val-Asp-Pro-Arg-Leu-Asp-Pro- | SEQ ID NO: 14 |
| (f) | -Val-Asp-Pro-Lys-Leu-Asp-Pro- | SEQ ID NO: 15 |
| (g) | -Val-Asp-Pro-Ser-Leu-Asp-Pro- | SEQ ID NO: 16 |
| (h) | -Val-Asp-Pro-Asn-Leu-Asp-Pro-. | SEQ ID NO: 17 |

As used herein, the term “HIV-1 Tat Epitope 2” refers to the sequences represented by the formula Lys-X-Leu-Gly-Ile-Ser-Tyr-Gly-Arg-Lys- SEQ ID NO: 5, wherein X is Gly or Ala. The entire scope of Epitope 2 sequences may be sequences of between 8 to about 10 amino acids in length within SEQ ID NO 5. For example, the sequence Leu-Gly-Ile-Ser-Tyr-Gly-Arg-Lys, amino acids 3-10 of SEQ ID NO: 5 may also serve as the sequence to which an antibody may bind. In another embodiment, Epitope 2 is the sequence Lys-Gly-Leu-Gly-Ile-Ser-Tyr-Gly-Arg-Lys, SEQ ID NO: 18. In still another embodiment Epitope 2 is the sequence Lys-Ala-Leu-Gly-Ile-Ser-Tyr-Gly-Arg-Lys SEQ ID NO: 19.

These definitions of Epitopes 1 and 2 of HIV Tat also encompass homologous or analogous modified epitope sequences, wherein the non-variable amino acids in the formula of SEQ ID NO: 5 or 6 (i.e., those not represented by a single letter and subscript) may be conservatively replaced individually by amino acid residues having similar characteristics. For example, the non-variable amino acid residues of SEQ ID NO: 5 or 6 may be replaced by other amino acid residues bearing the same charge and/or similar side chain lengths. Similarly the non-variable naturally-occurring amino acids in the SEQ ID NO: 5 or 6 may be replaced by unnatural amino acid residues, i.e., an amino acid having a modification in the chemical structure, e.g., a D-amino acid, an amino acid bearing a non-naturally occurring side chains an N-methylated amino acid, etc. See, e.g., L. Aurelio et al, 2002 Organic Letters, 4(21):3767-3769 and references cited therein, incorporated by reference herein.

As used herein, the term “ligand” refers to any protein that binds to another reference protein, such as a receptor protein. Thus, an anti-Epitope 2 ligand is any protein, whether an antibody or an engineered protein, that binds to the reference protein Epitope 2, as defined herein.

As used herein, the term “antibody” refers to an intact immunoglobulin having two light and two heavy chains. Thus a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, an Fv construct, a Fab construct, a light chain variable or complementarity determining region (CDR) sequence, or any sequence or construct that contains a sequence of the antibody that permits it to bind to its desired epitope, etc.

As used herein, the term “antibody” may also refer, where appropriate, to a mixture of different antibodies. For example, reference to an anti-Epitope 2 antibody includes reference to a single antibody or antibody fragment, or to a mixture of different antibodies. Such differences may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. As another example, mixtures of antibodies may be generated by antibodies that bind Epitope 2 when X is Gly and antibodies that bind to Epitope 2 when X is Ala.

Similarly, an “Epitope 1 antibody” as used herein may be an antibody or mixture of antibodies, each antibody specifically binding to a different Epitope 1 variant sequence, as represented by the variable amino acids in SEQ ID NO: 6. Alternatively, an Epitope 1 antibody refers to a single antibody or antibody fragment that binds to more than two such epitope sequences. Still another Epitope 1 antibody refers to an antibody or antibody fragment that specifically binds to more than five Epitope 1 variant sequences. In yet another embodiment, the anti-Epitope 1 antibody or fragment binds the epitope of SEQ ID NO: 6, in which R₁ is Val and R₂ is absent. In still another embodiment, the anti-Epitope 1 antibody or fragment binds at least one of the Epitope 1 variant sequences wherein Y₉ is Glu and at least one of the variant sequences wherein Y₉ is Asp. Still a further embodiment of a suitable anti-Epitope 1 antibody is one which binds at least one of the variant sequences selected from the group consisting of (a) through (d) and at least one of the variant sequences selected from the group consisting of (e) through (h) as listed above. In another embodiment, the Epitope 1 antibody of the invention can be a single antibody or a mixture of different antibodies, specifically binding to multiple variant HIV-1 Epitope 1 sequences having Glu in position Y₉ and at least one variant HIV-1 Epitope 1 sequence having Asp in position Y₉. Thus, a single Epitope 1 antibody or fragment useful in the present invention binds to HIV-1 Tat protein from multiple strains and subtypes. Preferably, the Epitope 1 antibody or fragment or mixture thereof binds to greater than 95% of the known HIV-1 strains and subtypes, including strains and subtypes from both B and non-B clades.

Such antibodies or fragments of Epitopes 1 and 2 useful in the method of this invention may be generated synthetically or recombinantly, using conventional

Each sample was added to a plate coated with the immobilized capture antibody to Epitope 1 as used in Example 1, or a mixture of anti-Epitope 1 antibodies, at 2 μg/ml. After incubation of the plasma samples with the bound anti-Epitope 1 antibody, the plate was washed and the detector antibody, i.e., the anti-Epitope 2 antisera used in Example 1 (previously incubated with the corresponding basic protein), was applied with a goat-anti-rabbit IgG, labelled with HRP, to provide the signal for the immobilized sandwich.

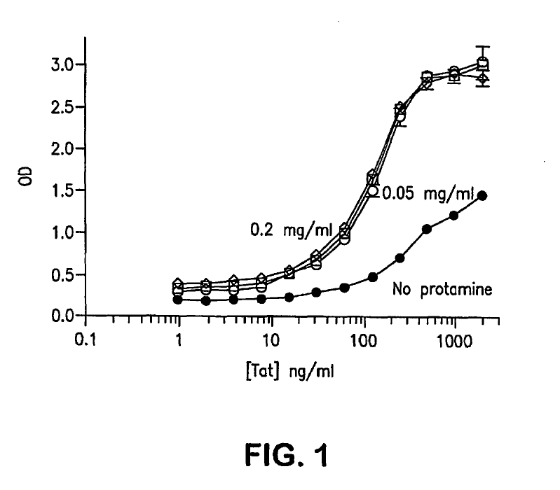

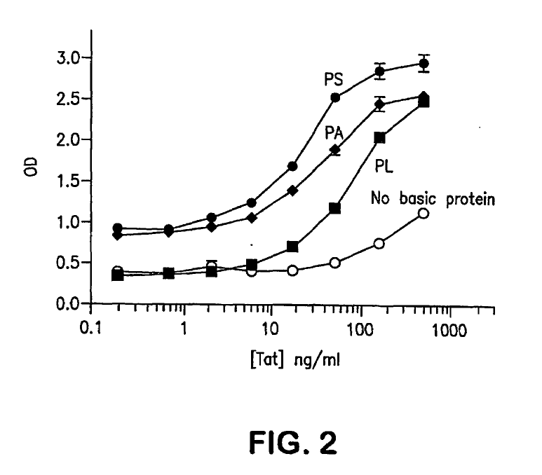

The resulting dose response curves are shown in FIG. 2. As with FIG. 1, the more Tat in the sample, the more signal was generated due to increased binding of the sandwiched IgG complex to the plate in all cases. More sensitive detection was obtained using the basic protein incubation step to neutralize acidic proteins present in the plasma that appear to interfere with binding of the antibody to Epitope 2. The data further showed that basic proteins other than protamine sulfate were useful in this assay, with protamine sulfate being the most active at the indicated concentration.

### EXAMPLE 3

### Comparative Results of Assay for Measuring Levels of HIV-1 Tat Vs. Detection of Viral RNA

The following example provides preliminary data comparing the results of the sandwich assay of the present invention to HIV-1 viral RNA detection by conventional methods. Twenty (20) uninfected control serum samples and 23 asymptomatic HIV-1 infected subject serum samples were tested by the method of this invention to measure HIV-1 Tat levels. In addition serum samples for three patients before, and/or during initial infection were tested in parallel with the results of viral DNA, using the Roche Monitor™ PCR assay (employed to detect first viral RNA in the same samples for comparison).

The sandwich assay of this invention was conducted by adding 60 μg/ml protamine sulfate to all control and infected samples for an hour at room temperature. The capture and detector antibodies and the protocol were as described for Example 2.

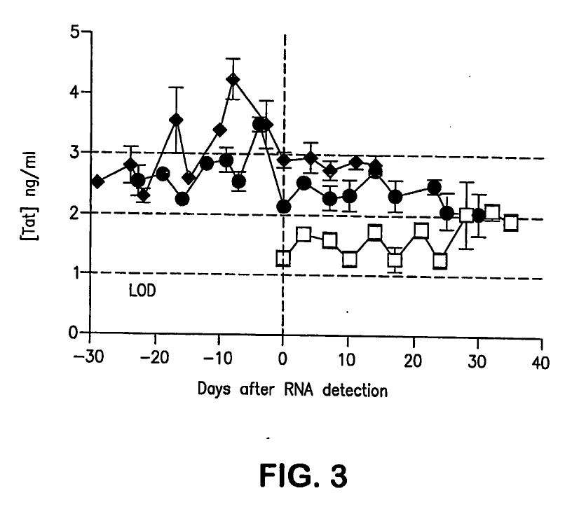

The data was recorded in graphical form in FIG. 3. All controls and 20 of the infected serums recorded Tat concentration at less than 1 ng/ml, i.e., below the limit of detection. Serum samples from two of the asymptomatic subjects were obtained prior to first detectable viral RNA. As indicated in the graph, these two patients showed detectable levels of Tat in serum at least 20 days before first detectable viral RNA was observed using the PCR assay. Timepoint 0 was the first detection of viral RNA.

Subsequently HIV RNA levels in the three indicated patients were plotted vs. days after first detectable RNA (day 0). Two subjects showed increasing viral load typical of acute infection. Results for a third subject lacking earlier serum samples were typical of somewhat later infection, indicated by initial decline of viral load.

In still another assay of this invention (data not shown) performed as described in the Example 2 above, Tat was detected in 19/46 (41%) of asymptomatic HIV plasmas and in only 5/30 (17%) control plasmas (i.e., false positives). This is a statistically significant detection of HIV-1 Tat in the asymptomatic subjects (P<0.03, Fishers Exact 2×2 test). The LOD for this assay was 0.2 ng/ml and the LOQ was 0.84 ng/ml. All plasma Tat protein levels in asymptomatic HIV-1 infected patients were below 1 ng/ml.

These examples demonstrate the utility and advantages of the claimed invention. All documents cited above are incorporated herein by reference.

1. A method for detecting or measuring HIV-1 Transactivating (Tat) protein in a biological sample comprising introducing into an assay reagent and/or said sample an amount of a basic protein effective to prevent acidic proteins or other acidic substances within said sample from interfering with binding between a ligand to Tat Epitope 2 and the Tat in the sample.

2. The method according to claim 1, wherein said ligand to Tat Epitope 2 is an antibody or antibody fragment that binds to HIV-1 Tat protein within the Epitope 2 amino acid sequence Lys-X-Leu-Gly-Ile-Ser-Tyr-Gly-Arg-Lys- of SEQ ID NO: 5, wherein X is Gly or Ala.

3. The method according to claim 2, comprising the steps of
(a) contacting said biological sample with said anti-Epitope 2 antibody and an amount of a basic protein effective to reduce interference with binding between said anti-Epitope 2 antibody and said Tat by acidic proteins within said sample; and
(b) determining the presence or amount of HIV-1 Tat in said sample by the presence or amount of binding between said Anti-Epitope 2 antibody and said Tat in said sample.

4. The method according to claim 1, wherein said basic protein is selected from the group consisting of protamine sulfate, poly-Arg, and poly-Lys.

5. The method according to claim 1, wherein said biological sample is plasma or serum.

6. The method according to claim 3, wherein said contacting step further comprises contacting said sample with an immobilized capture antibody to complex with any HIV-1 Tat in said sample, and contacting said sample containing capture antibody with complexed Tat with a detector antibody, which binds said immobilized capture antibody-HIV-1 Tat complex at a different HIV-1 Tat epitope than that bound by said capture antibody.

7. The method according to claim 6, further comprising the steps of:
(a) contacting said biological sample, said capture antibody and said detector antibody with a basic protein to neutralize the acidic proteins in said sample that interfere with binding between an HIV-1 Tat Anti-Epitope 2 antibody or antibody fragment that binds within the amino acid sequence Lys-X-Leu-Gly-Ile-Ser-Tyr-Gly-Arg-Lys- SEQ ID NO: 5, wherein X is Gly or Ala, and said Tat;
(b) contacting said biological sample (a) from a subject infected with HIV-1 with an immobilized capture antibody or antibody fragment that binds HIV-1 Tat at a first epitope present in said sample, wherein first immobilized complexes are formed between said capture antibody and said Tat in said sample; and
(c) contacting said first immobilized complexes of sample (a) with a detector antibody that binds HIV-1 Tat at a second epitope that is different from the epitope of said capture antibody, wherein second immobilized complexes are formed between said first immobilized complexes and said detector antibody;
wherein one of said capture antibody or detector antibody is said Tat anti-Epitope 2 antibody or antibody fragment; and
(d) detecting the amount of second immobilized complexes in said sample, wherein the amount of HIV-1 Tat in said sample is proportional to the amount of said second complexes.

8. The method according to claim 6, wherein said anti-Epitope 2 antibody is the capture antibody.

9. The method according to claim 6, wherein said capture antibody or detector antibody is an Epitope 1 antibody or fragment that binds an HIV-1 Tat Epitope 1 within the formula

| | |
|---|---|
| R₁-Asp-Pro-X₇-Leu-Y₉-Pro-R₂, | SEQ ID NO: 6 |

wherein X₇ is Arg, Lys, Ser or Asn;
wherein Y₉ is Glu or Asp;
wherein R₁ is absent or Val or Glu-Val; and
wherein R₂ is absent or Trp-Z₁₂-R₃,
wherein Z₁₂ is Lys or Asn, and
wherein R₃ is absent or is all or part of the sequence -His-Pro-Gly-Ser-.

10. The method according to claim 9, wherein said Epitope 1 antibody comprises multiple different antibodies to said Epitope 1.

11. The method according to any of claims 9, wherein said Epitope 1 and Anti-Epitope 2 antibody or antibodies are individually selected from the group consisting of a high affinity polyclonal antibody, a monoclonal antibody, a synthetic antibody, a recombinant antibody, a chimeric antibody, a humanized antibody, a human antibody, an isolated single antibody chain, or a fragment of said antibodies or antibody chain.

12. The method according to claim 8, wherein said detector antibody is an Epitope 1 antibody.

13. The method according to claim 6, wherein said contacting step comprises adding said basic protein to one or more of the following components of said assay:
(a) to said sample before contacting said sample with said antibody;
(b) to said capture antibodies before adding said antibodies to said sample;
(c) to said detector antibodies before adding said antibodies to said sample; and
(d) to each of (a) through (c).

14. The method according to claim 6, further comprising detecting the amount of immobilized capture antibody-Tat-detector antibody complexes in said sample, wherein the amount of HIV-1 Tat in said sample is proportional thereto.

15. A kit for use in performing an assay on a biological sample comprising:
(a) an amount of a basic protein effective to prevent acidic proteins or other acidic substances within said sample from interfering with binding between a ligand to Tat Epitope 2 and the Tat in the sample; and
(b) a ligand to Tat Epitope 2.

16. The kit according to claim 15, wherein said ligand is an Epitope 2 antibody or antibody fragment that binds to an HIV-1 Tat epitope within the amino acid sequence Lys-X-Leu-Gly-Ile-Ser-Tyr-Gly-Arg-Lys-SEQ ID NO: 5, wherein X is Gly or Ala.

17. The kit according to claim 15, further comprising a second antibody or antibody fragment that binds to an HIV-1 Tat epitope different from that of said Epitope 2 antibody.

18. The kit according to claim 15, further comprising one or more detectable labels or label systems for identifying binding of a detecting antibody to HIV-1 Tat present in said sample.

19. The kit according to claim 16, wherein said second antibody is selected from the group consisting of
(a) an antibody or a antibody fragment which binds to an HIV-1 Tat Epitope 1 variant sequence within the formula

| | |
|---|---|
| R₁-Asp-Pro-X₇-Leu-Y₉-Pro-R₂, | SEQ ID NO: 6 |

wherein X₇ is Arg, Lys, Ser or Asn;
wherein Y₉ is Glu or Asp;
wherein R₁ is absent or Val or Glu-Val; and
wherein R₂ is absent or Trp-Z₁₂-R₃,
wherein Z₁₂ is Lys or Asn, and
wherein R₃ is absent or is all or part of the sequence -His-Pro-Gly-Ser-; and
(b) a mixture of multiple different said Epitope 1 antibodies,
wherein said Epitope 1 antibody or fragment or mixture thereof binds to multiple variant HIV-1 Tat proteins from multiple strains and subtypes.

20. The kit according to claim 19, wherein said Epitope 2 and Epitope 1 antibodies are individually selected from the group consisting of a high affinity polyclonal antibody, a monoclonal antibody, a synthetic antibody, a recombinant antibody, a chimeric antibody, a humanized antibody, a human antibody, an isolated single antibody chain, or a fragment of said antibodies or antibody chain.
